(19) United States
(12) Patent Application Publication
(10) Pub. No.: US 2017/0275295 A1
(43) Pub. Date: Sep. 28, 2017

(54) METHOD FOR CONDITIONING A DIANHYDROHEXITOL, AQUEOUS SOLUTION OF CONDITIONED DIANHYDROHEXITOL, AND USES THEREOF

(71) Applicant: Roquette Freres, Lestrem (FR)

(72) Inventors: Mathias Ibert, La Chapelle d' Armentieres (FR); Hervé Wyart, Cuinchy (FR); Gwenaëlle Parent, Calonne Sur La Lys (FR)

(73) Assignee: Roquette Freres, Lestrem (FR)

(21) Appl. No.: 15/512,504

(22) PCT Filed: Sep. 18, 2015
(86) PCT No.: PCT/FR2015/052511

§ 371 Date: Mar. 17, 2017

(30) Foreign Application Priority Data
Sep. 19, 2014 (FR) 1458868

(51) Int. Cl. C07D 493/04; B32B 27/32

(52) U.S. Cl. CPC C07D 493/04; B32B 27/32

(57) ABSTRACT

The invention relates to a method for conditioning a dianhydrohexitol in the form of an aqueous solution in a container comprising a polyolefin-based layer. The invention also relates to the aqueous solution of thus-conditioned dianhydrohexitol, and to all the uses thereof, in particular those intended for food and pharmaceutical purposes.

The present invention relates to a method for conditioning a dianhydrohexitol, in the form of an aqueous solution in a packaging material comprising at least one layer based on polyolefin.

This is an extremely simple method in its implementation, and especially much less complex than the methods of the prior art which are essentially reserved for solid products, and which recommend using packaging materials which are impermeable to gases, and working under reduced oxygen and nitrogen pressure.

The method according to the present invention also proves to be inexpensive, which is another advantage. In addition, the applicant company has demonstrated that only a dianhydrohexitol in the form of an aqueous solution—and not a solid product—was able to be preserved in a stable manner in this type of packaging. As said packaging contains little, and preferentially does not contain any, antioxidant, a method is thus made available which makes it possible to package a dianhydrohexitol for applications in which it is sought to minimize—or even to exclude—antioxidants, such as the pharmaceutical and food sectors.

The present invention also relates to the aqueous solution of dianhydrohexitol packaged in this way, and all the uses thereof, especially including those in the food and pharmaceutical fields.

Dianhydrohexitols, also referred to as isohexides, are products of the internal dehydration of hydrogenated C6 sugars (hexitols) such as sorbitol, mannitol and iditol. Among these doubly dehydrated hydrogenated sugars, isosorbide is currently the one for which most industrial applications are envisioned, especially in the sector of plastic materials, as intermediate in chemical synthesis, but also in the food and pharmaceutical fields.

For the majority of these applications, it is generally necessary to have compositions which are as pure as possible, having especially a content of dianhydrohexitols at least equal to 98.5% by weight, preferably at least equal to 99.5% by weight relative to the total dry matter thereof. Now, dianhydrohexitols and in particular isosorbide are extremely hygroscopic products which are chemically not very stable. The applicant company has in particular observed that the storage of isosorbide manufactured according to known methods, even sheltered from atmospheric moisture, could lead, under certain temperature conditions, to chemical degradation giving rise, inter alia, to the formation of formic acid, which is an acid which has a characteristic unpleasant odor, which is particularly bothersome in pharmaceutical or other applications.

The applicant company was thus motivated to develop methods for the purification and stabilization of dianhydrohexitols, described especially in the patent applications EP 1 287 000 and WO 03/043959. The inventions relating to these applications cover both solid dianhydrohexitols and dianhydrohexitols in liquid form. A stable liquid composition of isosorbide with a dry matter content of between 50% and 90% is disclosed in the document WO 03/043959. As such, only the stability of solid products was evaluated in these two applications, by storage of test specimens in a container respectively made of plastic in polyethylene bags (EP 1 287 000) and made of glass (WO 03/043959) at a temperature equal to 60° C. and 40° C., respectively.

Subsequently, the applicant company noticed that the durations for preserving the solid dianhydrohexitols, determined under the conditions of the tests described in patent applications EP 1 287 000 and WO 03/043959, gave only a poor reflection of the stability of these same products under real conditions of transport and storage. The applicant company noted, in particular in certain cases, relatively large concentrations of formic acid close to the polyethylene film conventionally serving to package dianhydrohexitols in solid form, and especially isosorbide in the powder state.

The applicant company then observed that by substantially increasing the concentration of antioxidants in the layer of plastic material in contact with the dianhydrohexitol, the stability thereof was substantially improved, especially when the dianhydrohexitol in question was isosorbide. This invention was protected in patent application WO 2009/019371, the plastic packaging material being selected from polyethylene, polypropylene and copolymers of ethylene and of propylene.

It should be noted that the method for packaging which is the subject of said application applies to liquid or solid dianhydrohexitols, even if the solid form is favored, and that a most preferred variant consists in packaging the dianhydrohexitol under an anhydrous and/or inert atmosphere, for example under a nitrogen atmosphere. This document stipulates that, in order to guarantee optimum stability of the dianhydrohexitol during transport and storage, it is necessary to provide an additional layer of protection against oxygen in the air, steam and/or light.

In addition, it is worth noting that this type of solution does not make it possible to condition dianhydrohexitols for food or pharmaceutical applications, which both require heavily reduced amounts of antioxidants. By way of example, the FDA (Food and Drug Agency) produces an official list of the only antioxidants and stabilizers authorized for these two applications, with all the corresponding restrictions in terms of amounts, as a function of the polymer material in which they are incorporated.

Independently of this food or pharmaceutical context, the technical solutions developed in the preceding invention proved to require the development of complex conditioning materials and only applied to small amounts (approximately ten grams) of dianhydrohexitols, the problem of compacting of the dianhydrohexitols during their storage in bags of several kilograms or even several tens of kilograms remaining unsolved.

The latter problem was solved in the patent application WO 2013/021126 through the development of a method for packaging dianhydrohexitol in a conditioning material impermeable to gases, characterized in that the oxygen partial pressure within the conditioning is between 0.1 mbar and 10 mbar. A preferred variant consists in also limiting the nitrogen partial pressure within the conditioning to between 240 and 1012.9 mbar. It is indeed mentioned that these types of conditioning are suitable both for dianhydrohexitols in liquid form and solid form.

That being said, the document WO 2012/042187 indicates that for dianhydrohexitols in solid form, even if the latter are purified, stabilized and conditioned according to the techniques described above, it is still necessary to shape them into pellets to be entirely certain that the phenomenon of caking will be avoided, which phenomenon is particularly bothersome in operations of transport, handling and especially transferring of the products.

If the path of studies by the applicant company, as laid out above, is followed, the current prevailing solution for stable conditioning over time of the dianhydrohexitols therefore consists in using materials impermeable to gases, under reduced oxygen pressure, or even also under limited nitrogen pressure. It is readily appreciated that such an approach is both long and costly. Moreover, for dianhydrohexitols in powder form, it is suitable to provide, in addition to the abovementioned operations, a step of pelletizing with a view to avoiding problems of caking.

Now, and against all expectations, the applicant company has been able to observe, following numerous studies, that there is a very simple way to condition dianhydrohexitols in an extremely stable manner over time. This method relies on the conditioning of said dianhydrohexitol in the form of an aqueous solution in a packaging material comprising at least one layer based on polyolefin, such as, especially, polyethylene. Surprisingly, the applicant has observed that the stability of a dianhydrohexitol conditioned in liquid form in this way is much better than that of a solid dianhydrohexitol packaged according to the prior art cited above.

This is all the more surprising since this type of material did not give good results in the case of solid isosorbide, as indicated in the comparative examples of the document WO 2009/019371 already cited. Moreover, this also goes against the teaching according to which it is necessary to rely on conditioning materials impermeable to gases, under reduced oxygen pressure, or even also under limited nitrogen pressure.

Moreover and entirely advantageously, by limiting the amount of antioxidants in the packaging material, food and pharmaceutical applications become feasible, which was not certain in the case of the document WO 2009/019371 which recommends working with at least 0.1% by weight of these additives in the packaging barrier layer.

This is therefore a conditioning method which is particularly simple to implement, very inexpensive and perfectly suited to aqueous solutions of dianhydrohexitol. This method makes it possible especially to package and condition aqueous solutions of dianhydrohexitol in an extremely stable manner over time. In the present application, this stability is understood very simply through monitoring the change in the pH of said composition over time. A significant change or shift in this pH is proof of the phenomenon of oxidation of the dianhydrohexitol, which will lead to the formation of formic acid. Finally, dianhydrohexitols are advantageously made available, which may be packaged, handled and transported within the context of final applications in the food and pharmaceutical sectors.

The method which is the subject of said application therefore relates to a method for conditioning a dianhydrohexitol, comprising:
providing an aqueous solution of dianhydrohexitol,
introducing said aqueous solution of dianhydrohexitol into a container,
then closing said container,
   in which said container comprises a layer based on polyolefin.

The expression “aqueous solution of dianhydrohexitol” denotes a composition containing essentially water and at least one dianhydrohexitol. It is especially characterized by the dry matter thereof, expressed as % dry weight relative to the total weight thereof. The latter is especially between 40% and 95%, preferentially between 50% and 90%, very preferentially between 60% and 85%. It is moreover suitable not to associate said aqueous solution of dianhydrohexitol with another liquid form of dianhydrohexitol: namely a melt of dianhydrohexitol, that is to say a dianhydrohexitol in a molten state at a temperature of greater than or equal to 63+−2° C. (at atmospheric pressure).

Generally, dianhydrohexitols are synthesized in the presence of water (or water is generated during their synthesis): by recovering said dianhydrohexitol in this reaction medium, this immediately gives an aqueous solution of dianhydrohexitol which can be used according to the invention. The solutions of dianhydrohexitols may especially be obtained according to the methods described in the abovementioned patent applications EP 1 287 000 and WO 03/043959. It is possible to choose to retain all or some of the water used during the preparation of the dianhydrohexitol or to eliminate all the water to obtain a product in solid form which will be returned to aqueous solution by simply adding water, which constitutes another possibility for preparing an aqueous solution of dianhydrohexitol which can be used according to the invention.

The aqueous solution in question may contain a single dianhydrohexitol, just as it may contain several thereof. These dianhydrohexitols (1,4:3,6-dianhydrohexitols) encompass isosorbide (1,4:3,6-dianhydrosorbitol), isomannide (1,4:3,6 dianhydromannitol), isoidide (1,4:3,6-dianhydroiditol) and mixtures of at least two of these products. The aqueous solution preferably only contains a single dianhydrohexitol, which is isosorbide.

“Method for conditioning” is intended to mean an operation which consists in placing a product in a container, with which it is in direct contact, in order to facilitate the protection and preservation thereof.

The term “layer” denotes, in the present application, a uniform casing; this layer being “based on” polyolefin, which means that it consists predominantly of said polyolefin (said polyolefin then representing at least 90% by weight, preferentially 99%, very preferentially 100% by weight of the total weight of said “layer”).

The polyolefin may be selected in particular from polyethylene, polypropylene and copolymers of ethylene or of propylene. It is preferably polyethylene, more preferentially high-density polyethylene (HDPE).

HDPE is intended to mean a polyethylene with a molecular mass by weight of greater than 20 000 g/mol and having a melting range of between 90 and 160° C. This polymer may be of natural white color or may be colored by means of a dye, for example a blue dye, as long as this dye is approved for contact with food. It may also be of black color by addition of carbon black, with the proviso that it does not contain more than 0.1% by weight thereof relative to the total weight thereof.

Preferably, the container consists of a single layer, that is to say that it consists of a monolayer material, which is therefore based on polyolefin. It may nonetheless comprise other layers, that is to say consist of a multilayer material containing a layer based on polyolefin and at least one other layer, especially a layer based on an ethylene/vinyl alcohol copolymer (EVOH), on poly(vinylidene chloride) (PVDC), on polyamide (PA), on polyacrylonitrile (PAN), on poly(glycolic acid) (PGA), or a layer based on aluminum and/or on another suitable metal, deposited for example on the outer surface of a bag, or a sheet of aluminum and/or of another suitable metal.

According to one embodiment, the layer based on polyolefin does not contain more than 0.1% by weight of antioxidant relative to the total weight thereof. Preferentially, it does not contain more than 0.01% by weight thereof relative to the total weight thereof, and most preferably, it does not contain any antioxidant.

The term “antioxidant” covers, in the present application, exactly the same meaning as in the document WO 2009/019371, namely it encompasses all the compounds capable of limiting or of suppressing thermooxidative degradation, also known by the term autoxidation, of organic compounds, in particular of organic polymers.

A non-exhaustive list of these compounds is given in chapter 1, entitled “Antioxidants”, of the fifth edition of the work “Plastics Additives Handbook” (2001), Carl Hanser Verlag, Munich (Germany). When an antioxidant is optionally present in the polyolefin layer according to the present invention, it will preferentially be selected from the hydrogen donors such as secondary aromatic amines and highly sterically hindered phenols, the decomposition agents of hydroperoxides based on phosphorus such as phosphites and phosphonites, and those based on sulfur such as 3,3-thiodipropionic acid esters; and free-radical scavengers such as carbon black, highly sterically hindered amines, hydroxylamines, benzofuranone derivatives and phenols modified by acryloyl groups.

The overall thickness of the container does not play a decisive role in the present invention. It may especially be between 50 μm and 1 cm. The container is preferably a rigid material so as to be able to withstand the stresses linked to the packaging of a product in liquid form such as the aqueous solution according to the present invention. It may be a barrel, a container, a tank, but more generally it is thus a rigid container of any shape and size whatsoever. It may also be a flexible packaging, preferably held in a rigid structure making it possible to withstand the stresses linked to the packaging of a product in liquid form, for example a “bag-in-box” or a “flexitank”.

The method according to the present invention thus comprises introducing, into the container described above, the aqueous solution of dianhydrohexitol described above. This introduction is carried out by any means well known to those skilled in the art, especially means suited to the handling and transfer of products in liquid form, especially of compositions of dianhydrohexitol in liquid form. These means are, for example, gravity filling from a storage tank located above the packaging material by means of a filling orifice, or by means of pumping equipment to transfer the aqueous solution of dianhydrohexitol from the tank to the packaging material.

The container is then closed. This closure takes place by virtue of devices known to those skilled in the art and adapted to the shape of the material in question. These devices are for example the closure of the filling orifice by means of a screw cap or crimping by means of a crimper.

Another subject of the present invention relates to an aqueous solution of dianhydrohexitol conditioned in a container, said container comprising a layer based on polyolefin.

In other words, the invention relates to a container which contains, or in which is placed, an aqueous solution of dianhydrohexitol, said container comprising a layer based on polyolefin.

In this context, the aqueous solution of dianhydrohexitol, on the one hand, and the layer based on polyolefin, on the other, assume all the features listed above relating to the method which constitutes the first subject of the present application.

The content of dry matter of the aqueous solution of dianhydrohexitol is especially between 40% and 95%, preferentially between 50% and 90%, very preferentially between 60% and 85% relative to the total weight thereof. As explained above, this aqueous solution does not cover what is referred to as a melt.

The aqueous solution may contain a single dianhydrohexitol, just as it may contain several thereof. These dianhydrohexitols (1,4:3,6-dianhydrohexitols) encompass isosorbide (1,4:3,6-dianhydrosorbitol), isomannide (1,4:3,6 dianhydromannitol), isoidide (1,4:3,6-dianhydroiditol) and mixtures of at least two of these products. The aqueous solution preferably only contains a single dianhydrohexitol, which is isosorbide.

With regard to the container, the polyolefin may be selected in particular from polyethylene, polypropylene or copolymers of ethylene and of propylene. It is preferably polyethylene, more preferentially high-density polyethylene (HDPE).

HDPE is intended to mean a polyethylene with a molecular mass by weight of greater than 20 000 g/mol and having a melting range of between 90 and 160° C. This polymer may be of natural white color or may be colored by means of a dye, for example a blue dye, as long as this dye is approved for contact with food. It may also be of black color by addition of carbon black, with the proviso that it does not contain more than 0.1% by weight thereof relative to the total weight thereof.

Preferably, the container consists of a single layer, that is to say that it consists of a monolayer material, which is therefore based on polyolefin. It may nonetheless consist of other layers, that is to say consist of a multilayer material containing a layer based on polyolefin and at least one other layer, especially a layer based on an ethylene/vinyl alcohol copolymer (EVOH), on poly(vinylidene chloride) (PVDC), on polyamide (PA), on polyacrylonitrile (PAN), on poly(glycolic acid) (PGA), or a layer based on aluminum and/or on another suitable metal, deposited for example on the outer surface of a bag, or a sheet of aluminum and/or of another suitable metal.

According to one embodiment, the layer based on polyolefin does not contain more than 0.1% by weight of antioxidant relative to the total weight thereof. Preferentially, it does not contain more than 0.01% by weight thereof relative to the total weight thereof, and most preferably, it does not contain any antioxidant.

The overall thickness of the packaging material does not play a decisive role in the present invention. It may especially be between 50 μm and 1 cm. The packaging material is preferably a rigid material so as to be able to withstand the stresses linked to the packaging of a product in liquid form such as the aqueous solution according to the present invention. It may be a barrel, a container, a tank, but more generally it is thus a rigid container of any shape and size whatsoever. It may also be a flexible packaging, preferably held in a rigid structure making it possible to withstand the stresses linked to the packaging of a product in liquid form, for example a “bag-in-box” or a “flexitank”.

A final subject of the present invention relates to the use of the abovementioned aqueous solution of dianhydrohexitol in the manufacture of plastic, as intermediate in chemical synthesis, in the food and pharmaceutical industries, and more preferentially in the food and pharmaceutical industries.

The invention will be better understood in the light of the following examples which are in no way limiting.

### EXAMPLES

The following examples illustrate the benefit of the packaging method according to the invention, by demonstrating that it enables packaging of a composition of dianhydrohexitol (isosorbide in the case in point) which remains very stable over time, as evidenced by the change in pH thereof.

Not only does the pH of the liquid composition of isosorbide remain stable over several months, regardless of the storage conditions, but it is even more stable than the pH of the same composition of isosorbide in solid form.

### Example 1

This example relates to the packaging and storage of isosorbide, in solid or liquid form, said isosorbide having been stabilized beforehand with disodium phosphate.

The procedure begins first of all by manufacturing a solid isosorbide composition and a liquid isosorbide composition, in the following way:

1 kg of a solution of sorbitol at 70% of dry matter sold by the applicant company under the name Neosorb 70/02 and 7 g of concentrated sulfuric acid are introduced into a jacketed stirred reactor. The mixture obtained is heated under vacuum (pressure of approximately 100 mbar) for 5 hours so as to eliminate the water contained in the initial reaction medium and that originating from the sorbitol dehydration reaction.

The reaction crude is then cooled to 100° C. and then neutralized with 11.4 g of a 50% (by weight) sodium hydroxide solution. The isosorbide composition neutralized in this way is then distilled under vacuum (pressure lower than 50 mbar).

The slightly colored (light yellow color) crude isosorbide distillate is then dissolved in 2-propanol, at a temperature of 60° C., so as to obtain a solution with 75% DM. This solution is then cooled slowly, over the course of 5 hours, down to a temperature of 10° C. A recrystallized isosorbide seed is added at 40° C.

The crystals are then drained in a centrifuge and washed with 2-propanol. After drying under vacuum, the crystals are redissolved in water so as to obtain a DM of 40%.

This solution is then percolated on a column of granular active carbon CPG 12-40 at a rate of 0.5 BV/h (Bed Volume/hour). The decolored isosorbide composition thus obtained is then passed, at a rate of 2 BV/h, successively over a column of Purolite C 150 S strong cationic resin and then a column of Amberlite IRA 910 strong anionic resin. This solution is then treated with powdered active carbon of Norit SX+type at 20° C. for 1 hour. The active carbon is used in a proportion of 0.5% expressed by dry weight/dry weight of solution.

0.005% of disodium phosphate (dry weight/dry weight of isosorbide contained in the composition) is then introduced into said composition.

After filtration, the isosorbide solution is concentrated under vacuum. The solution is concentrated until a solution with 80% dry matter is obtained. A portion of the solution is recovered in order to carry out tests 4 and 5. The concentration under vacuum of the remainder of the solution is then carried out, in order to eliminate the residual water. The molten mass obtained crystallizes on cooling in the form of a massed product of large crystals which is subsequently ground to obtain a white-colored powder having a moisture content of 0.2%. This powder is recovered for tests 1, 2 and 3.

These different compositions then serve to illustrate the packaging method according to the invention or according to the prior art.

### Test No. 1

This test relates to the prior art and illustrates the packaging of the solid isosorbide composition in a “PE+Alu” conditioning. More specifically, this conditioning consists of a first inner bag (20 cm×20 cm) made of polyethylene (PE) with a thickness of 100 μm, combined with a second outer bag (25 cm×25 cm) consisting of an aluminum complex (Alu) containing 80 μm thick polyethylene covered with 8.5 μm thick aluminum.

The isosorbide composition is packaged in the following manner: 100 g of the solid isosorbide composition as obtained above are introduced into the inner PE bag which is closed by sealing using an impulse heat sealer (SZ 380 model sold by Joisten & Kettenbaum GmbH & Co, Bergisch Gladbach, Germany). This bag is itself placed inside the outer bag Alu, which is then closed by sealing with the same heat sealer in order to ensure leaktightness with regard to the outer atmosphere.

### Test No. 2

This test relates to the prior art and illustrates the packaging of the solid isosorbide composition in a conditioning consisting of “white PE BigBag Liner/alu/PET”. More specifically, this conditioning consists of a bag (25 cm×25 cm) consisting of a complex of total thickness of approximately 100 μm consisting of an inner layer made of polyethylene (PE) with a thickness of 80 μm, an intermediate layer made of aluminum with a thickness of 9 μm and an outer layer made of polyethylene terephthalate (PET) with a thickness of 12 μm.

The isosorbide composition is packaged in the following manner: 100 g of the solid isosorbide composition as obtained above are introduced into the “white PE BigBag Liner/alu/PET” bag which is closed by sealing using an impulse heat sealer (SZ 380 model sold by Joisten & Kettenbaum GmbH & Co, Bergisch Gladbach, Germany) in order to ensure leaktightness with regard to the outer atmosphere.

### Test No. 3

This test relates to the prior art and illustrates the packaging of the solid isosorbide composition in a conditioning consisting of “carbon PE+Alu”. More specifically, this conditioning consists of a first inner bag (20 cm×20 cm) made of carbon-additivated polyethylene (carbon PE) with a thickness of 150 μm, and a second, outer, bag (25 cm×25 cm) consisting of an aluminum complex (Alu) containing 80 μm thick polyethylene covered with 8.5 μm thick aluminum.

The isosorbide composition is packaged in the following manner: 100 g of the solid isosorbide composition as obtained above are introduced into the inner carbon PE bag which is closed by sealing using an impulse heat sealer (SZ 380 model sold by Joisten & Kettenbaum GmbH & Co, Bergisch Gladbach, Germany). This bag is itself placed inside the outer bag Alu, which is then closed by sealing with the same heat sealer in order to ensure leaktightness with regard to the outer atmosphere.

### Test No. 4

This test relates to the invention and illustrates the packaging of the liquid isosorbide composition in a “white HDPE” conditioning. More specifically, this conditioning is a natural color 150 ml HDPE flask not containing antioxidants or dyes.

The isosorbide composition is packaged in the following manner: 100 g of the liquid isosorbide composition as obtained above are poured into the white HDPE flask which is closed by screwing on a cap consisting of the same material in order to ensure leaktightness with regard to the outer atmosphere.

### Test No. 5

This test relates to the invention and illustrates the packaging of the liquid isosorbide composition in a “black HDPE” conditioning. More specifically, this conditioning is a black color 150 ml HDPE flask containing less than 0.1% of black dye.

The isosorbide composition is packaged in the following manner: 100 g of the liquid isosorbide composition as obtained above are poured into the black HDPE flask which is closed by screwing on a cap consisting of the same material in order to ensure leaktightness with regard to the outer atmosphere.

The solid and liquid isosorbide compositions packaged according to tests 1 to 5 are placed in a ventilated oven, thermostated at the temperature of 50° C. Several conditionings per test are placed in the oven in order to monitor the change in pH of each composition over time.

The change in pH of each composition over time is monitored in the following way: firstly, for each solid or liquid isosorbide composition, all the sample is extracted from the conditioning materials and is added to osmosed water in order to obtain a solution of isosorbide with 40% dry matter in osmosed water, then the initial pH of this solution is measured. The pH measurement is carried out on a pH meter of Radiometer Analytical PHM 220 brand equipped with a combined Ag/AgCl wire electrode of Mettler Toledo brand, calibrated beforehand using pH 7 and 4 buffer solutions. After a determined period of storage at 50° C., a solution of isosorbide with 40% dry matter is prepared in the same way in osmosed water for each solid or liquid isosorbide composition, then the pH is measured using the same pH meter.

The results are given in table 1. They demonstrate clearly that the aqueous solution of isosorbide conditioned according to the invention in a “black HDPE” and “white HDPE” packaging is much more stable than the solid isosorbide conditioned in the “PE+Alu”, “white PE BigBag Liner/alu/PET” and “carbon PE+Alu” packaging.

TABLE 1

| Test No. | 1 | 2 | 3 | 4 | 5 |
|---|---|---|---|---|---|
| Packaging | White PE + | White PE | Carbon | White | Black |
| | Alu | BigBag | PE + Alu | HDPE | HDPE |
| | | Liner/alu/PET | | | |
| Physical | Solid | Solid | Solid | Liquid | Liquid |
| form | | | | | |
| pH | | | | | |
| 0 day | 7.2 | 7.6 | 7.2 | 7.4 | 7.4 |
| 1 month | 3.1 | 3.9 | 3.2 | 7.2 | 7.2 |
| 2 months | | | | 7.1 | 7.2 |
| 3 months | | | | 4.0 | 6.8 |

### Example 2

This example relates to the packaging and storage of isosorbide, in solid or liquid form, said isosorbide having been stabilized beforehand with diethanolamine.

The procedure begins first of all by manufacturing a liquid isosorbide composition and a solid isosorbide composition in the same way as for example 1, except for the fact that the disodium phosphate is replaced by 0.0025% of diethanolamine by dry weight relative to the dry weight of isosorbide.

The liquid and solid compositions obtained as above then serve to illustrate the packaging method according to the invention or according to the prior art.

### Test No. 6

This test relates to the prior art and illustrates the packaging of the solid isosorbide composition in a “PE+Alu” conditioning. More specifically, this conditioning consists of a first inner bag (20 cm×20 cm) made of polyethylene (PE) with a thickness of 100 μm, combined with a second outer bag (25 cm×25 cm) consisting of an aluminum complex (Alu) containing 80 μm thick polyethylene covered with 8.5 μm thick aluminum.

The isosorbide composition is packaged in the following manner: 100 g of the solid isosorbide composition as obtained above are introduced into the inner PE bag which is closed by sealing using an impulse heat sealer (SZ 380 model sold by Joisten & Kettenbaum GmbH & Co, Bergisch Gladbach, Germany). This bag is itself placed inside the outer bag Alu, which is then closed by sealing with the same heat sealer in order to ensure leaktightness with regard to the outer atmosphere.

### Test No. 7

This test relates to the prior art and illustrates the packaging of the solid isosorbide composition in a conditioning consisting of “white PE BigBag Liner/alu/PET”. More specifically, this conditioning consists of a bag (25 cm×25 cm) consisting of a complex of total thickness of approximately 100 μm consisting of an inner layer made of polyethylene (PE) with a thickness of 80 μm, an intermediate layer made of aluminum with a thickness of 9 μm and an outer layer made of polyethylene terephthalate (PET) with a thickness of 12 μm.

The isosorbide composition is packaged in the following manner: 100 g of the solid isosorbide composition as obtained above are introduced into the “white PE BigBag Liner/alu/PET” bag which is closed by sealing using an impulse heat sealer (SZ 380 model sold by Joisten & Kettenbaum GmbH & Co, Bergisch Gladbach, Germany) in order to ensure leaktightness with regard to the outer atmosphere.

### Test No. 8

This test relates to the invention and illustrates the packaging of the liquid isosorbide composition in a “white HDPE” conditioning. More specifically, this conditioning is a natural color 150 ml HDPE flask not containing antioxidants or dyes.

The isosorbide composition is packaged in the following manner: 100 g of the liquid isosorbide composition as obtained above are poured into the white HDPE flask which is closed by screwing on a cap consisting of the same material in order to ensure leaktightness with regard to the outer atmosphere.

### Test No. 9

This test relates to the invention and illustrates the packaging of the liquid isosorbide composition in a “black HDPE” conditioning. More specifically, this conditioning is a black color 150 ml HDPE flask containing less than 0.1% of black dye.

The isosorbide composition is packaged in the following manner: 100 g of the liquid isosorbide composition as obtained above are poured into the black HDPE flask which is closed by screwing on a cap consisting of the same material in order to ensure leaktightness with regard to the outer atmosphere.

The solid and liquid isosorbide compositions packaged according to tests 6 to 9 are placed in a ventilated oven, thermostated at the temperature of 50° C. Several conditionings per test are placed in the oven in order to monitor the change in pH of each composition over time.

The change in pH of each composition over time is monitored according to the same protocol as for example 1.

The results are given in table 2. As for example 1, it is observed that the aqueous solutions of isosorbide packaged according to the invention are much more stable than the solid compositions of isosorbide packaged according to the prior art.

TABLE 2

| Test No. | 6 | 7 | 8 | 9 |
|---|---|---|---|---|
| Packaging | White PE + | White PE | White | Black |
| | Alu | BigBag | HDPE | HDPE |
| | | Liner/alu/PET | | |
| Physical form | Solid | Solid | Liquid | Liquid |
| pH | | | | |
| 0 day | 8.1 | 8.4 | 8.2 | 8.2 |
| 1 month | 6.5 | 7.5 | 7.7 | 8.2 |
| 2 months | 3.2 | 7.5 | 7.6 | 8.2 |
| 3 months | | 3.2 | 7.2 | 7.4 |

1. A method for conditioning a dianhydrohexitol, comprising:
providing an aqueous solution of dianhydrohexitol,
introducing said aqueous solution of dianhydrohexitol into a container,
then closing said container,
in which said container comprises a layer based on polyolefin.

2. The method as claimed in claim 1, wherein said layer based on polyolefin does not contain more than 0.1% by weight of antioxidant relative to the total weight thereof.

3. The method as claimed in claim 1, wherein the polyolefin is selected from polyethylene, polypropylene or copolymers of ethylene and of propylene, and is preferentially polyethylene, more preferentially high-density polyethylene (HDPE).

4. The method as claimed in claim 1, wherein the container also contains at least one layer based on an ethylene/vinyl alcohol copolymer (EVOH), on poly(vinylidene chloride) (PVDC), on polyamide (PA), on polyacrylonitrile (PAN), on poly(glycolic acid) (PGA), on aluminum and/or on another metal.

5. The method as claimed in claim 1, wherein the dianhydrohexitol is selected from isosorbide, isomannide, isoidide or mixtures of at least two of these products, and is preferentially isosorbide.

6. The method as claimed in claim 1, wherein that the aqueous solution of dianhydrohexitol has a content of dry matter of between 40% and 95%, preferentially between 50% and 90%, very preferentially between 60% and 85% relative to the total weight thereof.

7. A container containing an aqueous solution of dianhydrohexitol, said container comprising a layer based on polyolefin and said layer not containing more than 0.1% by weight of antioxidant relative to the total weight thereof.

8. The container containing an aqueous solution of dianhydrohexitol as claimed in claim 7, wherein the polyolefin is selected from polyethylene, polypropylene or copolymers of ethylene and of propylene, and is preferentially polyethylene, more preferentially high-density polyethylene (HDPE).

9. The container containing an aqueous solution of dianhydrohexitol as claimed in claim 7, wherein the container also contains at least one layer based on an ethylene/vinyl alcohol copolymer (EVOH), on poly(vinylidene chloride) (PVDC), on polyamide (PA), on polyacrylonitrile (PAN), on poly(glycolic acid) (PGA), on aluminum and/or on another metal.

10. The container containing an aqueous solution of dianhydrohexitol as claimed in claim 1, wherein the dianhydrohexitol is selected from isosorbide, isomannide, isoidide or mixtures of at least two of these products, and is preferentially isosorbide.

11. The container containing an aqueous solution of dianhydrohexitol as claimed in claim 7, wherein said solution of dianhydrohexitol has a content of dry matter of between 40% and 95%, preferentially between 50% and 90%, very preferentially between 60% and 85% relative to the total weight thereof.
